(19) United States
(12) Patent Application Publication
(10) Pub. No.: US 2010/0062022 A1
(43) Pub. Date: Mar. 11, 2010

(54) IMMUNOGENIC PROTEINS OF BURKHOLDERIA PSEUDOMALLEI AND USES THEREOF

(76) Inventors: Sarah Victoria Harding, Wiltshire (GB); Mitali Sarkar-Tyson, Wiltshire (GB); Petra Claire Farquar Oyston, Wiltshire (GB); Richard William Titball, Wiltshire (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/376,474

(22) PCT Filed: Aug. 7, 2007
(86) PCT No.: PCT/GB07/02989

§ 371 Date: Feb. 5, 2009

(30) Foreign Application Priority Data
Aug. 7, 2006 (GB) 0615635.0

(51) Int. Cl. A61K 39/02; C07K 14/195; C07K 16/12; G01N 33/569; A61P 31/04; A61P 37/04

(52) U.S. Cl. 424/234.1; 530/350; 530/387.1; 435/7.32

(57) ABSTRACT

A protein derived from an outer layer of Burkholderia pseudomallei or a fragment or a variant of said protein, wherein the protein, fragment, or variant is capable of producing a protective immune response in an animal, for use in the treatment of infection by Burkholderia species. Pharmaceutical compositions comprising such proteins are also described and claimed.

This invention relates to the detection and identification of Burkholderia species, and to providing medicaments, vaccines and other treatments suitable for the prophylactic and therapeutic treatment of infections caused by Burkholderia species.

Burkholderia pseudomallei, the causative agent of the disease melioidosis, is endemic in South East Asia and Northern Australia, where it can be commonly found in soil and stagnant water. The disease it causes in humans is variable, ranging from acute septicaemia to a chronic or latent infection. It is reported to have a mortality rate of 50% in North East Thailand. The treatment of infected patients is complex due to the intrinsic resistance of the bacterium to antibiotics. It has been reported that death occurs in as many as 40% of patients who receive antibiotics post-infection

Burkholderia mallei is the causative agent of glanders, an equine disease which can be transmitted to humans with fatal consequences. Although incidences of glanders in the Western world are relatively low, cases do still occur in Asia, Africa, South America, and Central America.

Burkholderia cepacia is also an opportunistic environmental pathogen, and though it is virtually non-pathogenic in healthy patients, it causes respiratory infection in cystic fibrosis patients and occasionally nosocomial infection in patients in intensive care units

There is, currently, no available vaccine for treatment of Burkholderia infections.

As a result, there is a clear requirement to develop prophylactic and therapeutic treatments for Burkholderia infections. There is considerable research activity focused in this area. Ideally, a single vaccine which protects against all virulent strains of Burkholderia species is required.

The prior art shows that some cell surface components have already been investigated as potential subunit vaccines. For example, Nelson et al (Journal of Medical Microbiology (2004), 53, 1177-1182) shows that lipopolysaccharide (LPS) and capsular polysaccharide are protective in the mouse model of infection. However, protection was not complete. The need remains, therefore, to find alternative vaccines which are capable of providing more effective protection against Burkholderia infections than those already proposed. Any alternative vaccine would, ideally, be completely protective against the most virulent strains of Burkholderia such that a single vaccine is effective against all strains of the bacterium.

Accordingly the present invention provides proteins which are immunogenic and protective against infection by Burkholderia species, such as B. mallei and B. pseudomallei.

In a first aspect the present invention provides a protein derived from an outer layer of Burkholderia pseudomallei or a fragment or a variant of said protein, wherein the protein, fragment, or variant is capable of producing a protective immune response in an animal, for use in the treatment of infection by Burkholderia species.

As used herein the terms “outer layer protein”, “surface layer protein” and “outer membrane protein” may be used interchangeably and mean a protein which is present, partially or completely, on a bacterial cell surface and includes proteins which are permanently or generally located on the cell surface and also proteins which are exported from the bacterium to the cell surface occasionally, for example when the cell is under stress, or temporarily, for example during a particular phase of the life cycle of the cell.

As used herein the expression “capable of producing a protective immune response” means that the substance is capable of generating a protective immune response in a host organism such as a mammal for example a human, to whom it is administered.

As used herein the terms “protein” and “polypeptide” are used interchangeably and mean a sequence of amino acids joined together by peptide bonds. The amino acid sequence of the polypeptide is determined by the sequence of the DNA bases which encode the amino acids of the polypeptide chain.

As used herein the term “fragment” refers to any portion of the given amino acid sequence of a polypeptide or protein which has the same activity as the complete amino acid sequence. Fragments will suitably comprise at least 5 and preferably at least 10 consecutive amino acids from the basic sequence and does include combinations of such fragments. In order to retain activity, fragments will suitably comprise at least one epitopic region. Fragments comprising epitopic regions may be fused together to form a variant.

In the context of the present invention the expression “variant” as used herein refers to sequences of amino acids which differ from the base sequence from which they are derived in that one or more amino acids within the sequence are substituted for other amino acids. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. Suitably variants will be greater than 75% identical, preferably at least 80% identical, more preferably at least 85% identical, and most preferably at least 90% identical to the base sequence. Variants included in the description of the present invention are intended to exclude substitutions which result in the variant having a substantially identical sequence to a genomic sequence from another organism.

Identity in this instance can be judged for example using the BLAST program (vs. 2.2.12) found at http://www.ncbi.nlm.nih.gov/BLAST/ or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The bacterial outer membrane of B. pseudomallei has been found to provide an interface for host-pathogen interactions and harbours proteins that have a variety of important roles including the adherence to and invasion of host cells, resistance to phagocytosis and the degradation of host cells. Surface associated proteins also play a role in maintaining structural integrity and in the adaptation of bacterial pathogens to differing environments within the host. As they are exposed and accessible to the host's immune system, outer membrane proteins (OMP's) can make good vaccine candidates.

Specific proteins which are protective and useful in the treatment of infection include, but are not limited to those proteins, protective fragments and protective variants of those proteins listed in Table 1, below. Preferred proteins include BPSS0839 (SEQ ID no1), BPSS1850 (SEQ ID No 2), BPSS0213 and BPSS1679 and protective fragments and variants of each of these proteins.

TABLE 1
Immunogenic and Immunoreactive proteins derived from B. pseudomallei.

| | GenBank | | | Homology to | Homology to |
|---|---|---|---|---|---|
| Protein | Accession | | | B. mallel 23344 | B. thaliandensis |
| (locus) | No. | Functional Class | Chromosome | (%) | 264 (%) |
| DnaK | 53720436 | Cell processes | 1 | 91 | 90 |
| Pnp | 53718843 | Macromolecule | 1 | 95 | 94 |
| | | degradation | | | |
| BPSS1850 | 53722869 | Conserved in | 2 | 91 | 85 |
| | | organisms other than | | | |
| | | B. pseudomallei | | | |
| BPSS0839 | 53721865 | Conserved in | 2 | 94 | 91 |
| | | organisms other than | | | |
| | | B. pseudomallei | | | |
| GroEL | 53720307 | Cell processes | 1 | 90 | 90 |
| BPSS1679 | 53722699 | Membrane or | 2 | 94 | 78 |
| | | exported | | | |
| PhaP | 53729908 | Not classified | 1 | 88 | 87 |
| Tuf | 53720836 | Macromolecule | 1 | 89 | 89 |
| | | synthesis/modification | | | |
| BPSS0879 | 53721903 | Membrane or | 2 | 87 | 87 |
| | | exported | | | |

The proteins are preferably derived from a virulent strain of B. pseudomallei such as B. pseudomallei strain K96243. Such proteins are particularly useful in treating or preventing infection caused by B. mallei or B. pseudomallei, or B. cepacia. As such, the proteins may be formulated into pharmaceutical compositions which may be used to treat infection.

According to a second aspect, the present invention provides a pharmaceutical composition comprising a protein derived from an outer layer of Burkholderia pseudomallei or a fragment or a variant of said protein, wherein the protein, fragment, or variant is capable of producing a protective immune response in an animal, in combination with a pharmaceutically acceptable carrier or excipient.

The pharmaceutical composition preferably comprises a protein or a protective fragment or a protective variant of the proteins listed in Table 1 above. Suitably these proteins (or fragments or variants) will be derived from B. pseudomallei strain K96243.

Suitable excipients and carriers which may be used in the pharmaceutical compositions will be known to those skilled in the art. These may include solid or liquid carriers. Suitable liquid carriers include water or saline. The polypeptides and proteins of the composition may be formulated into an emulsion or alternatively they may be formulated in, or together with, biodegradable microspheres or liposomes. Suitably the composition further comprises an adjuvant which stimulates the host's immune response. Particularly suitable adjuvants include Alhydrogel, MPL+TDM and Freunds Incomplete Adjuvant.

In a preferred embodiment, the composition further comprises an additional protective antigen which is protective against infection by Burkholderia species. The additional antigen may be another protein or polypeptide which is selected from the group described in Table 1, but preferably the additional protective antigen is another protein or polypeptide which has been shown to be protective against Burkholderia infection. Examples of such additional protective antigen include those described in co-pending international patent application PCT/GB2006/001354, the contents of which are incorporated herein by reference. Preferably the additional protective antigen is a protein selected from the group of proteins consisting of OppA, PotF and LoIC derived from Burkholderia pseudomallei and protective fragments and protective variants of these proteins. It is more preferred that the additional protective antigen is LoIC or a protective fragment of variant of LoIC. Such pharmaceutical compositions are particularly useful for the treatment of melioidosis or glanders.

Proteins of the invention are particularly useful for raising antibodies, which are useful in the detection of Burkholderia species. The fact that the proteins are derived from the outer surface layers of B. pseudomallei is useful in that it allows for rapid detection of B. pseudomallei or B. mallei, without the need for extensive culturing of the cells.

Accordingly, in a third aspect of the invention there is provided an antibody or a binding fragment thereof, which binds specifically to a protein as hereinbefore described.

Antibodies or binding fragments thereof may be polyclonal or monoclonal, which may be produced using conventional methods.

For instance, polyclonal antibodies may be generated by immunisation of an animal (such as a rabbit, rat, goat, horse, sheep etc) with immunogenic proteins or immunogenic subunits or fragments thereof, to raise antisera, from which antibodies may be purified.

Monoclonal antibodies may be obtained by fusing spleen cells from an immunised animal with mycloma cells, and selecting hybridoma cells which secrete suitable antibodies.

Antibody binding fragments include F(ab′)2, F(ab)2, Fab or Fab′ fragments, as well as recombinant antibodies, such as single chain (sc) antibodies FV, VH or VK fragments, but they may also comprise deletion mutants of an antibody sequence. Acronyms used here are well known in the art. They are suitably derived from polyclonal or monoclonal antibodies using conventional methods such as enzymatic digestion with enzymes such as papain or pepsin (to produce Fab and F(ab′)2 fragments respectively). Alternatively, they may be generated using conventional recombinant DNA technology.

These antibodies may be conveniently incorporated into any available antibody based assay, which is optimised for the detection of Burkholderia species. Similarly the antibodies are also useful for the diagnosis of melioidosis and glanders by incorporating them into serodiagnostic assays. Suitable antibody based assays can be readily determined by person skilled in the art.

Accordingly, a method of detecting the presence of B. pseudomallei or B. mallei represents a fourth aspect of the present invention. Such a method comprises contacting a sample suspected of containing B. pseudomallei or B. mallei cells with an antibody raised against any of the proteins as hereinbefore described, or a binding fragment of said antibody, and detecting binding therebetween.

Detection methods used include conventional immunological methods for example ELISA, surface plasmon resonance and the like.

The sample is suitably an environmental sample, suspected of containing B. mallei or B. pseudomallei cells. Suitably the antibody or binding fragment is immobilised on a solid support, for example on an ELISA plate, but other forms of support, for example membranes such as those utilised in conventional “dip-stick” tests may also be employed.

Detection of a complex between a surface layer protein within in the sample, and a binding moiety as described above can be detected using conventional methods, in particular immunological methods such as ELISA methods. Assay formats may take various forms including “sandwich” or “competitive” types.

In a typical sandwich assay, the binding moiety is immobilised on a support, such as an ELISA plate, where is it contacted with a sample suspected of containing B. mallei or B. pseudomallei cells. Where present, these cells will bind the binding moiety and so become immobilised in their turn. The support is then separated from the sample, for example by washing. The presence of the cells on the support can then be detected by application of secondary antibodies or binding fragments thereof, which bind to the cells, and are detectable, for example because they are labelled for instance with a visible label such as a fluorescent label, or a radiolabel, but preferably that they can be developed to produce a visible signal: A particular example of a secondary antibody is an antibody, or binding fragment, that carries an enzymatic label, such as horseradish peroxidase, which can then be utilised to produce a signal by addition of the enzyme substrate, using conventional ELISA methodology. Secondary antibodies used in this way may also comprise binding moieties in accordance with the invention.

In a particular competitive assay format, the binding moiety of the invention is immobilised on a support. In this instance, a protein which binds said binding moiety in competition to the cells is added to the sample prior to contact with the support. Any cells present within the sample will compete with this protein for binding to the immobilised binding moiety. Thus, the absence of peptide on the support is indicative of the presence of cells in the sample.

In this case, the competing protein is suitably labelled so that it may be readily detected, for instance using a visible label such as a fluorescent or radiolabel. Alternatively, it may be detected using a secondary antibody or a binding fragment thereof, such as those discussed above in relation to sandwich assays, which binds the protein.

According to a fifth aspect, the present invention provides a protein derived from an outer layer of Burkholderia pseudomallei or a fragment or a variant of said protein, wherein the protein, fragment, or variant is capable of producing a protective immune response in an animal. Examples of suitable proteins include, but are not limited to BPSS0839 (SEQ ID no 1), BPSS0879 (SEQ ID no 2), BPSS1679 (SEQ ID no 3), BPSS1850 (SEQ ID no 4), BPSS0213 and fragments and variants of these proteins. Particular examples of the proteins are BPSS0839 (SEQ ID no 1), BPSS1850 (SEQ ID no 2). These proteins have not previously been identified as outer surface proteins in B. pseudomallei or B. mallei and the present inventors have now shown that these proteins are present on the outer surface of B. pseudomallei, are immunoreactive and immunogenic. These proteins are, therefore, preferred embodiments of the invention.

Proteins of the invention may be prepared using conventional methods. Although they may be isolated from B. pseudomallei, it is preferable that they are expressed recombinantly. For this purpose, a nucleic acid encoding the proteins is incorporated into an expression vector or plasmid, which is then used to transform a host cell. The host cell may be a prokaryotic or eukaryotic cell, but is preferably a prokaryotic cell such as E. coli. The codons utilised in the nucleic acid may be optimised for expression in the particular host cell.

Nucleic acids encoding novel proteins of the invention, as well as vectors and cells containing these form a further aspect of the invention.

Accordingly these proteins, or protective fragments or protective variants of these proteins are particularly useful in methods of preventing or treating infection caused by Burkholderia species, such as melioidosis and glanders.

The invention will now be particularly described by way of example with reference to the accompanying diagrammatic drawings where:

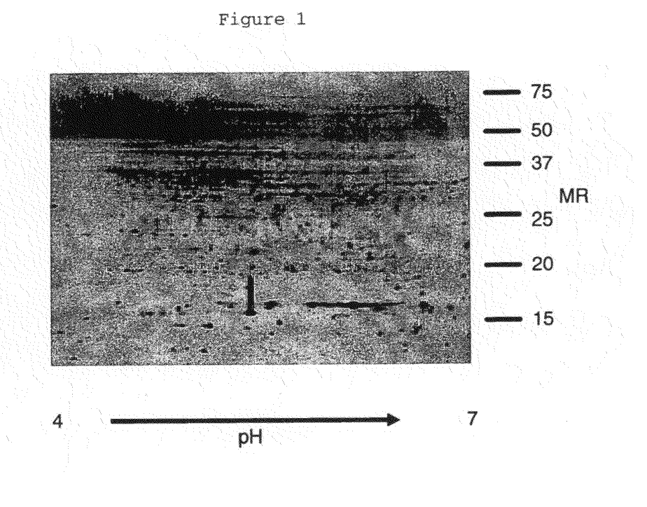

FIG. 1 shows a image of a 2 dimensional gel of a preparation enriched for outer membrane proteins from B. pseudomallei using a pH 4-7 IPG strip for IEF and a 12-14% gel for SDS-PAGE. Proteins were visualised using silver staining.

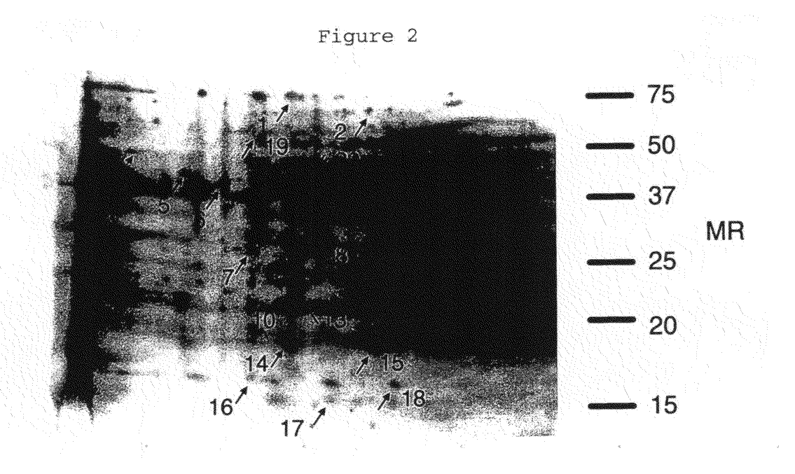

FIG. 2 shows a western blot of the biotinylated proteins from B. pseudomallei. The twenty proteins highlighted are some of those that reacted with streptavidin and subsequently identified.

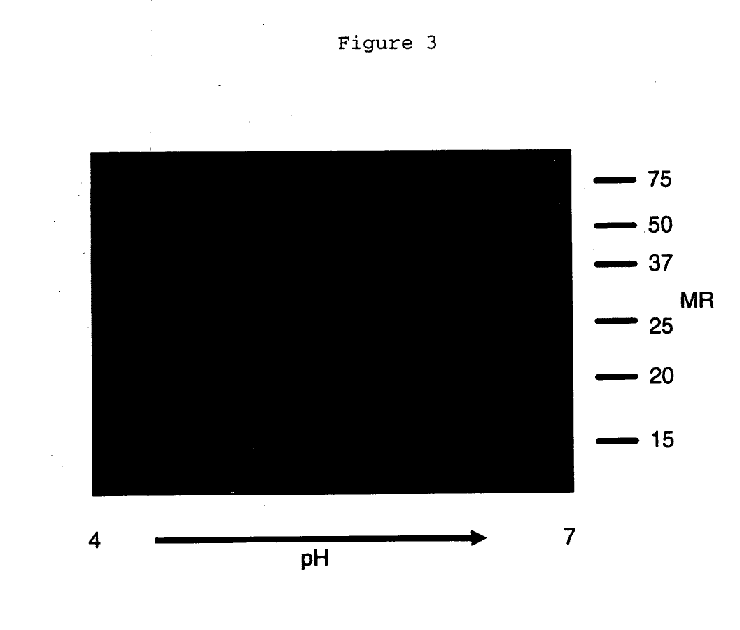

FIG. 3 shows a western blot of the immunoreactive proteins from B. pseudomallei. The 10 proteins highlighted are those that reacted with human convalescent sera, and were subsequently identified using mass spectrometry.

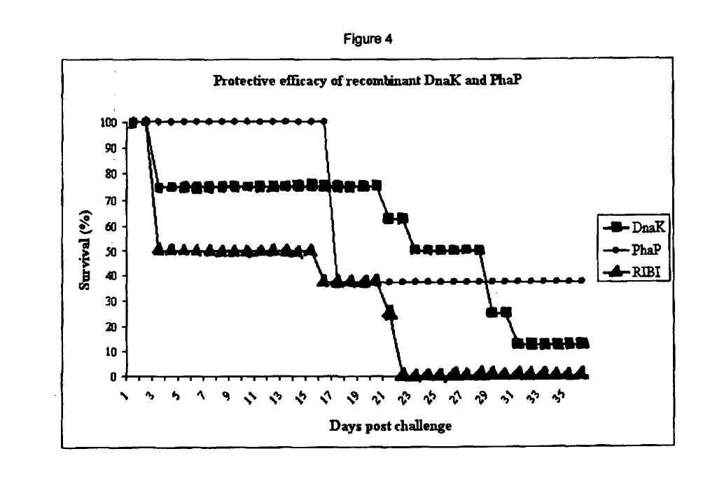

FIG. 4 shows a graph detailing numbers of survivors (mice) immunised with a protein according to the present invention (PhaP) following an intraperitoneal challenge with virulent B. pseudomallei strain K96243

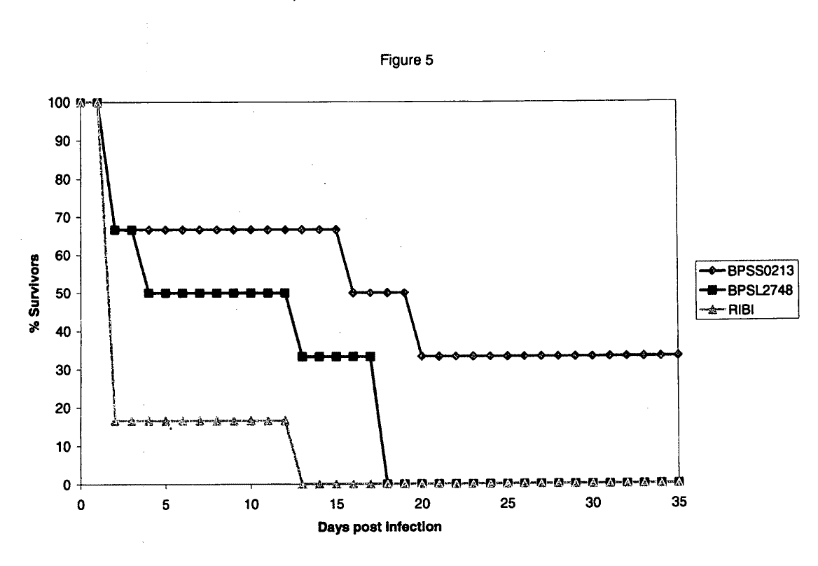

FIG. 5 shows a survival curve for 6 mice immunised with another protein of the present invention, BPS0213, and subsequently challenged with B. pseudomallei strain K96243.

### MATERIALS AND METHODS

### Bacterial Strains and Growth Conditions

B. pseudomallei strain K96243 obtained from S. Songsivilai, Siriraj Hospital, Thailand was grown in M9 minimal media supplemented with 2 mM iron sulphate with agitation overnight at 37° C. The cells were harvested at log phase (determined by an OD₆₀₀ reading of 0.5) for 15 min at 10,000 ×g.

### Human Convalescent Sera

All sera samples were obtained from Defence Science Organisation Laboratories, Singapore from patients diagnosed as having melioidosis. The clinical data obtained from these patients is shown in Table 2.

TABLE 2
Clinical data of melioidosis patients

| | | | | Indirect |
|---|---|---|---|---|
| | | | | Haemagglu- |
| | | | Sera | tination |
| Patient | | Predisposing | collection | Assay |
| Identity | Gender/Age | Risk Factors | point | (IHA) titre |
| 1 | Male/44 | Alcoholic, | After 14 days of | 1:256 |
| | | smoker | treatment | |
| 2 | Male/51 | Diabetes | After 18 days of | 1:1024 |
| | | mellitus, renal | treatment | |
| | | disease | | |
| 3 | Male/51 | Hypertension, | After 14 days of | 1:256 |
| | | renal disease | treatment | |
| 4 | Female/28 | None | Unknown | 1:64 |
| 5 | Male/56 | Diabetes | 6 weeks after | 1:128 |
| | | mellitus, | presentation | |
| | | hypertension, | | |
| | | smoker | | |
| 6 | Male/57 | Diabetes | 14 days after | 1:521 |
| | | mellitus | presentation | |
| 7 | Male/68 | Diabetes | 14 days after | 1:1024 |
| | | mellitus | presentation | |
| 8 | Male/47 | Unknown | On | 1:64 |
| | | | presentation | |

### Biotinylation of Whole Cells

The bacterial cells at a concentration of 10⁹ cfu/ml were washed three times in 50 ml Debecco's phosphate buffered saline (PBS) and harvested for 15 min at 10,000 g. The bacterial suspension was then incubated with EZ-Link Sulfo-NHS-LC-Biotin (HyClone) at a final concentration of 5 mg/ml for 1 hr at room temperature. The bacterial cells were pelleted by centrifugation at 10,000 g for 15 min and washed three times with 50 ml PBS.

### Preparation of Outer Membrane Proteins

The bacterial cells were harvested at 10,000 g for 15 min, and resuspended in 40 mM Tris (Solution 1, ReadyPrep Sequential Extraction kit, BioRad). Lysozyme was then added to a final concentration of 10 mg/ml and incubated at room temperature for 30 min. The bacterial suspension was then freeze thawed three times on dry ice. DNase and RNase was added at a concentration of 1 μg/ml and incubated at room temperature for 30 min. The sample was then centrifuged at 10,000 g for 30 min and the supernatant containing primarily hydrophilic proteins was removed.

The pellet was resuspended in 8M urea, 4% (w/v) 3-[3-(Cholamidopropyl)dimethylammonio]-1-proanesulfonate (CHAPS), 40 mM Tris, 0.2% (w/v) Bio-lyte 3/10 ampholyte, tributylphosphine (TBP), (Solution 2, ReadyPrep Sequential Extraction kit, BioRad) vortexed for 5 min and centrifuged at 10,000 g for 30 min. The supernatant containing primarily inner membrane proteins was removed.

The pellet was finally resuspended in 5M urea, 2M Thiourea, 2% (w/v) CHAPS, 2% (w/v) SB3-10, 40 mM Tris, 0.2% (w/v) Bio-lyte 3/10 ampholyte, TBP (Solution 3, ReadyPrep Sequential Extraction kit, BioRad) and centrifuged at 10,000 g for 30 min. The supernatant containing primarily hydrophobic proteins was then removed for analysis. The fractions were stored at −20° C. until required.

### 1D Gel Electrophoresis (1 DE)

1 DE was performed on Novex® 4-20% Tris-Glycine gels (Invitrogen) following a method developed by Laemmli (1970) and using a XCell SureLock™ Mini-Cell (Invitrogen). Gels were silver stained using the SilverQuest™ Silver Staining Kit (Invitrogen).

### Western Blotting of 10 Gels

Replicate 1D gels were transferred to nitrocellulose membrane using a XCell II™ Blot Module (Invitrogen) for 1.5 h at 125 V, then the membrane was blocked with 5% BSA for 1 h. The membrane was washed in PBS+0.05% tween (PBST) three times, then incubated with a 1:1000 dilution of HRP-conjugated streptavidin antibody (Amersham Biosciences) in PBST for 45 min (to identify biotinylated proteins) or with rabbit sera raised against heat killed B. pseudomallei K96243 (to identify immunoreactive proteins) (data not shown). The wash step was repeated three times, then the membrane was incubated with a 1:1000 dilution of HRP-conjugated goat anti-rabbit secondary antibody (Amersham BioSciences) in PBST for 45 min (to identify immunoreactive proteins). The wash step was repeated three times, then the membrane was placed into ECL Western blotting detection reagents (Amersham Biosciences), and manually developed with Kodak solutions.

### 2D Gel Electrophoresis (2DE)

Two hundred micrograms of the biotinylated extract enriched for OMP's was solubilised in 8 M urea, 2% CHAPS, 0.5% Immobilised pH gradient (IPG) buffer (Amersham BioSciences) and 3 mg/ml dithiothreitol (DTT). This was applied to an 18 cm pH 4-7 Immobiline strip (Amersham BioSciences), then rehydrated on a IPGphor machine (Amersham Biosciences) for 12 h. Isoelectric focusing (IEF) was then performed at 500 V for 500 Vh, 1000 V for 1000 Vh and 8000 V for 64,000-80,000 Vh. The IPG strip was then equilibrated in 6 M urea, 2% SDS, 50 mM Tris HCl, 30% glycerol, 70 mg/ml DTT (450 mg/ml iodoacetamide). The strip was then loaded onto an SDS-PAGE ExcelGel (12-14%, 1 mm thick) and run on a Multiphor II gel system (Amersham Biosciences) at 100 V, 20 mA and 40 W for 45 min then 1000 V, 40 mA and 40 W for 160 min.

Alternatively OMP's were separated as detailed in the ReadyPrep 2-D starter kit instruction manual (BioRad). This used PROTEAN IEF trays (BioRad) for the rehydration, an IPGPhor II machine (Amersham BioSciences) for the IEF and a PROTEAN II XL electrophoresis cell (BioRad) for the separation of proteins. Two gels were run at 32 mA for 30 min, then 75 mA for 3 h. Gels were silver stained using the PlusOne silver stain kit (Amersham Biosciences).

### Western Blotting of 2D Gels

Replicate 2D gels were transferred to nitrocellulose membrane in a Semi-dry transfer apparatus (BioRad or Amersham BioSciences) for 1.5 h at 200 mA, and then the membrane was blocked with 5% BSA for 1 h. The membrane was washed in PBS+0.05% tween (PBST) three times, then incubated with a 1:1000 dilution of HRP-conjugated streptavidin antibody (Amersham Biosciences) in PBST for 45 min (to identify biotinylated proteins) or with pooled human sera (to identify immunoreactive proteins) (see Table 2). The wash step was repeated three times, then the membrane was incubated with a 1:5000 dilution of HRP-conjugated mouse anti-human IgG secondary antibody (Accurate Chemical and Scientific Corporation) in PBST for 45 min (to identify immunoreactive proteins). The wash step was repeated three times, then the membrane was placed into ECL Western blotting detection reagents (Amersham Biosciences), and manually developed with Kodak solutions.

### In-Gel Trypsin Digestion

Protein spots were excised from gels and destained with 30 mM potassium ferricyanide and 100 mM sodium thiosulphate (at a ratio of 1:1) based on the method by Gharandaghi et al, (1999). They were washed in 50% acetonitrile and 0.1 M ammonium bicarbonate and reduced and alkylated in 10 mM DTT and 55 mM iodoacetamide. Proteins were digested overnight with 12.5 ng/ml porcine trypsin (Promega) made up in digestion buffer (50 mM ammonium bicarbonate, 0.1 mM calcium chloride) at 37° C. and the peptides extracted using 5% formic acid and 5% acetonitrile (at a ratio of 1:1), based on a method developed by Shevchenko et al, (1996).

### Matrix Assisted Laser Desorption Ionisation Time of Flight (MALDI-TOF) Mass Spectrometry

The matrix used for MALDI was recrystallised α-hydroxycinnamic acid (HCCA). MALDI analysis was performed using a Bruker Ultraflex MALDI-TOF (Bruker Daltonics) with a 400 Å Anchorchip™ target plate (Bruker). 1 mg/ml HCCA in acetone was diluted 1:2 with ethanol and 1 μl mixed with 0.5 μl sample and crystallised on the target. Acquired spectra were analysed in FlexAnalysis and BioTools software (Bruker). Peptide mass fingerprints were searched using the program MASCOT, and the program PSORTb v.2.0 was used to predict the cellular location of identified proteins (Nakai, 1999). SignalP 3.0 was used to predict the presence of signal peptides (Bendtsen et al, 2004). Protein similarities were perfomed using Basic Local Alignment Search Tool (BLAST) (http://www.ncbi.nlm.nih.gov/BLAST) and the public NCBI database.

### Expression of Proteins (PhaP, DnaK and BPSS0213)

The proteins PhaP, DnaK and BPSS0213 were cloned as full length proteins with a His-tag in the pET vector system and expressed in E. coli BL21* cells. The recombinant proteins were purified using HiTrap columns and were then used in challenge experiments as fusion-proteins, i.e. without not cleavage of the His-tag.

### Protection Study—PhaP

The protective efficacy of recombinant PhaP and DnaK was assessed by the following methods: BALB/c mice were immunised three times with PhaP, DnaK or RIBI only. Five weeks following the last immunisation mice were challenged with 3×10⁴ cfu of B. pseudomallei strain K96243 by the intraperitoneal route. The animals were monitored for signs of disease for 5 weeks after which the surviving animals were culled.

### Protection Study—BPSS0213

The protective efficacy of recombinant BPSS0213 was assessed in the following way: Three doses of BPSS0213 were administered to 6 BALB/C mice, intraperintoneally with RIBI as adjuvant (10 μg per mouse per dose). Five weeks after the last immunisation, the mice were challenged intraperitoneally with 33 MLD B. pseudomallei K96243.

### Results

Visualisation of OMP's from B. pseudomallei

Outer membrane located proteins are known to have important roles in the pathogenesis of disease caused by many micro-organisms. A 200 μg extract of B. pseudomallei cells enriched in hydrophobic proteins was separated by 2DE. Approximately two hundred distinct proteins spots could be detected after silver staining (FIG. 1). Most of the spots were found to be in the pH 4-7 range and had molecular masses of 15-75 kDa.

### Identification of Biotinylated Proteins

Western blotting using an HRP-conjugated streptavidin antibody was used to identify the proteins that had been labelled with biotin (FIG. 2). Spots of interest were determined by overlay analysis of the silver stained 2D gels and streptavidin blots. Proteins corresponding to thirty five spots on the Western blots were excised from the gels, trypsin digested and subjected to mass spectrometry to obtain the molecular masses of the peptides. Proteins were identified by MALDI-TOF or MALDI-QTOF mass spectrometry using the protein search engine MASCOT (Matrix Science) to probe the NCBI non-redundant database

### Identification of Immunoreactive Proteins

Western blotting using human convalescent sera and mouse anti-human IgG as primary and secondary antibodies respectively were used to identify immunoreactive proteins (FIG. 3). Spots of interest were again determined by overlay analysis of the silver stained gels and Western blots and identified by mass spectrometry. Ten protein spots were identified from the 2D gels using human sera as a probe. Two other proteins were identified as reacting with rabbit sera raised against heat killed B. pseudomallei K96243 whole cells by 1 DE analysis. Proteins were identified as above.

### Functional Classification of Proteins

The identified proteins were assigned into functional classes loosely based on the Monica Riley classification system. The majority of the biotinylated proteins belonged to the following three classes: membrane or exported, (5 proteins), macromolecule synthesis or modification (5), and cell processes (3) (Table 3). The majority of immunoreactive proteins belonged to membrane or exported (3 proteins), energy metabolism (2) and cell processes (2) (Table 4). 4 proteins that were biotinylated and immunoreactive were found to be involved in cellular processes or were membrane or exported proteins (Table 6).

BLAST analysis was undertaken to determine whether these proteins are present in other closely related Burkholderia species: Burkholderia mallei strain 23344 and Burkholderia thailandensis strain 264. Of the biotinylated proteins identified 29 have 80% or more sequence homology with predicted proteins in B. mallei compared to 26 proteins in B. thailandensis strain 264 (Table 5). All proteins found to be immunoreactive have homologues in B. mallei and except for BPSS1679 are all present in B. thailandensis (Table 5). This is also the case for those proteins found to be both biotinylated and immunoreactive (Table 6).

### Cellular Location of Identified Proteins

PSORTb v.20 was used to predict the cellular location of all proteins identified. This analysis predicted 3 of the 35 biotinylated proteins identified to be outer membrane located, with 14 predicted as cytoplasmic, 17 of unknown location and 1 predicted as being inserted into the cytoplasmic membrane (Table 3). SignalP 3.0 software was also used to predict the presence of signal peptides. Six of the 35 biotinylated proteins were predicted as having a signal peptide.

PSORTb predicted 4 of 12 immunoreactive proteins identified as being outer membrane located, 5 predicted as cytoplasmic and 3 as having an unknown location (Table 4). Four of these proteins were predicted as having a signal peptide. Of the 9 proteins that were shown to be both biotinylated and immunoreactive, 3 were predicted as being outer membrane located, 3 cytoplasmic and 3 had an unknown location (Table 6). Four of these proteins were predicted as having a signal peptide.

### Protection Study—PhaP

The results of the protection study, showing the protective efficacy of PhaP are shown in FIG. 4. Mice immunised with PhaP were afforded better protection than mice immunised with adjuvant alone.

### Protection Study—BPSS0213

The protective effect of BPSS0213 is demonstrated in FIG. 5, which shows that mice immunised with BPSS0213 shows significant protection compared with RIBI control. Furthermore, bacteria were present in spleens, liver and lungs of all survivors

### Discussion

Surface layer proteins from B. pseudomallei were selected using an extract that had been enriched for outer membrane proteins and used two different proteomics based approaches in parallel, which provides greater confidence in the results achieved.

Biotin labelling of proteins and their subsequent detection using avidin is a known method of protein identification. Biotin selectively labels the lysine residues of proteins exposed on the cell surface and should not penetrate the membrane. 35 proteins were identified by this method with differing roles within the cell, the two main protein groups being membrane located or exported proteins or those involved with macromolecular synthesis or modification.

PSORTb v.2.0 was used to predict the cellular localisation of all identified proteins. Theoretically all biotinylated proteins identified should be membrane located although some periplasmic proteins may have been labelled due to permeation of the cell membrane pumps or labelling after secretion. Additionally these proteins could be released during the biotinylation process or could have been purified during the outer membrane protein extraction. There is evidence that some proteins predicted by PSORT as being cytoplasm located are actually found on the cell surface so is therefore only used as a guide.

This technique identified many stress defence related proteins including superoxide dismutase (SodB) which has been identified as a virulence factor in Listeria monocytogenes and has been reported to induce protection against Brucella abortus and alkyl hydroperoxide reductase (AhpC) which when knocked out in Salmonella typhimurium increased sensitivity to killing by organic hydroperoxides and therefore virulence. These stress related proteins may be more abundant in these experiments as B. pseudomallei was grown in M9 minimal media to try to mimic the in vivo host environment where nutrients are not as widely available.

This approach was used in combination with the technique of Western blotting using human convalescent sera. B. pseudomallei induces a complex immune response which includes antibodies primarily to surface exposed antigens. In this way, twelve proteins were found to react with components of the sera, the majority belonging to the membrane or exported group of proteins.

Chaperones including GroEL and DnaK are regularly identified as being reactive with human sera. It has also been previously shown that GroEL is antigenic in B. pseudomallei and is actively secreted in Bartonella bacilliformis. It is thought that heat shock proteins themselves are not protective, but may have a role as carriers of foreign antigens in protecting against B. pseudomallei.

The multifunctional protein elongation factor Tu (EF-Tu) was identified as being biotinylated and reactive with rabbit sera. Although EF-Tu plays a role in protein synthesis it is also recognised as having chaperone-like properties in prokaryotes to have adhesive properties in Mycoplasma pneumoniae, and Lactobacillus Johnsonii potentially playing a role in virulence.

We have identified two porins as being both biotinylated and immunoreactive. Porins are membrane channels involved in nutrient transport and maintaining structural integrity. There is evidence that porins induce protection against bacterial pathogens. Immunising with PorB from Neisseria meningitis induced a bactericidal immune response against the organism. This bactericidal response was found to be sustained and lifelong when Salmonella enterica serovar Typhi porins OmpC and OmpF were used to immunise mice. These identified porins are therefore useful in providing a level of protection against B. pseudomallei.

Interestingly eight proteins found to be biotinylated and immunoreactive in B. pseudomallei were found to also be present with a homology of 80% or above in both pathogenic B. mallei and avirulent B. thailandensis. Those proteins present in both B. pseudomallei and B. mallei can, according to this invention, be used in the development of a sub-unit vaccine that cross-protects against both organisms as they are genetically very similar. In particular, one protein, (BPSS1679) encoding a porin protein was found by us to be present in B. pseudomallei and B. mallei but absent in B. thailandensis. The absence of this protein in B. thailandensis suggests that this porin may be required for the organism to become fully virulent and could play a role in causing disease in B. pseudomallei and B. mallei. Thus, in particular, this invention proposes use of this protein as a diagnostic target.

### SEQUENCES

(BPSS0839)

SEQ. ID no. 1

msyksilvhl dtsdrararl etaltlarqf gaylsavfav
ytpeptsfyv magtadyfad qqrrrdekra alerlfhael
kradvegqwi vadaraneav phyaryadlv iagqtdpddp
etyvddsfpd tlvlsagrpv lllpyagmps aigtrvlvaw
dgsreatrav hdaapflala tkttivtvng aaheppgari
pgadialtla rhhanidvrd lerardasig dvllshayes
gtdllvmgay gharwkelil ggvtrtifas mtvpvlmsh
(BPSS1850)

SEQ ID no. 2

mddhrriapp farrlhplsl llaaslahge tgappaerrs
dappatalap ifvtanplga salssptasl sgdaltlrrt
dslgdtlngl pgvstttygp lvgrpiirgm dgdrirllqn
gvaaydassl sydhavpqdp lsverieivr gpaallyggn
avggvvntid nripreaitg vsgaldasyg gannaragaa
lveggngrfa fhldafgret dalripghah sarqraldge
dasepygklp nsdgrrygga aggsytwadg yvgasysgye
snygsvaetd arlqmrqerv alasevrnlr gpfsqlkfdf
gytnyqhkei edgvtgttfr nhgyearvea rhrklgpfeg
algvqvgqnt fsalggeala pttrttsval fgleqwqatd
alklsagari ehvrldpsan gddkfgfars rdfnagsvsa
galyqlapaw slagnvsyte raptfyelya ngphgatgqy
ligrpdaqke kavstdlalr yasgpnrgsi gvfysrlrny
laeydtgrlv dddgvpvapg addalreavy rgvraefygv
elegrwrafe rrghrvdlel sadytharna dtgeplpria
plratlaady gygpfgaraq lthawaqhrv pehdlatdgy
tslgvvltyk lrvgatnwla ylrgdnltnq diryassvvr
niapqggrsv sigmrttf

1. A protein derived from an outer layer of Burkholderia pseudomallei or a fragment or a variant of said protein, wherein the protein, fragment, or variant is capable of producing a protective immune response in an animal, wherein the immune response is protective against infection by Burkholderia species.

2. The protein of claim 1 which is selected from the group of proteins consisting of BPSS0839, BPSS1850, BPSS0879, BPSS1679, BPSS0213, DnaK, Pnp, GroEL, PhaP, Tuf and fragments and variants thereof.

3. The protein of claim 1 which is derived from an outer layer of Burkholderia pseudomallei strain K96243 or a fragment or a variant thereof.

4. The protein of claim 1 wherein the infection is caused by B. mallei or B. pseudomallei, or B. cepacia.

5. The protein if claim 4 wherein the infection is caused by B. pseudomallei.

6. A pharmaceutical composition comprising a protein derived from an outer layer of Burkholderia pseudomallei or a fragment or a variant of said protein, wherein the protein, fragment, or variant is capable of producing a protective immune response in an animal, in combination with a pharmaceutically acceptable carrier or excipient.

7. The pharmaceutical composition of claim 6 wherein the protein is selected from the group of proteins consisting of BPSS0839, BPSS1850, BPSS0879, BPSS1679, BPSS0213, DnaK, Pnp, GroEL, PhaP, Tuf and fragments and variants thereof.

8. The pharmaceutical composition of claim 7 wherein the protein is BPSS1679.

9. The pharmaceutical composition of claim 6 wherein the protein is derived from an outer layer of Burkholderia pseudomallei strain K96243 or a fragment or a variant thereof.

10. The pharmaceutical composition of claim 6 which comprises an additional protective antigen which is protective against infection by Burkholderia species.

11. The pharmaceutical composition of claim 10 wherein the additional protective antigen is a protein selected from the group of proteins consisting of OppA, PotF and LoIC derived from Burkholderia pseudomallei and protective fragments and protective variants of these proteins.

12-15. (canceled)

16. An antibody or a binding fragment thereof which binds specifically to the protein of claims 1.

17. A method for detecting the presence of B. pseudomallei or B. mallei which method comprises contacting a sample suspected of containing B. pseudomallei or B. mallei cells with the antibody of claim 16, or a binding fragment of said antibody, and detecting binding therebetween.

18. A protein derived from an outer layer of Burkholderia pseudomallei or a fragment or a variant of said protein, wherein the protein, fragment, or variant is capable of producing a protective immune response in an animal.

19. The protein of claim 18 wherein the protein is selected from the group of proteins consisting of BPSS0839, BPSS1850, BPSS1679, BPSS0879, BPSS0213 and fragments and variants of these proteins.

20. The protein of claim 19 wherein the protein is BPSS1679.

21. An isolated nucleic acid which encodes the protective protein or protective fragment or protective variant of claim 18.

22. A method of preventing or treating infection in an animal caused by Burkholderia species which comprises administering an effective amount of the protein of claim 1 to the animal infected with Burkholderia species.

23. A method of preventing or treating infection in an animal caused by Burkholderia species which comprises administering an effective amount of the pharmaceutical composition of claim 6 to the animal infected with Burkholderia species.
